(19) United States
(12) Patent Application Publication
(10) Pub. No.: US 2014/0276347 A1
(43) Pub. Date: Sep. 18, 2014

(54) INTRAOSSEOUS SHUNTS

(71) Applicant: University of Rochester, Rochester, NY (US)

(72) Inventor: Jonathan J. STONE, Rochester, NY (US)

(73) Assignee: UNIVERSITY OF ROCHESTER, Rochester, NY (US)

(21) Appl. No.: 14/213,521

(22) Filed: Mar. 14, 2014

Related U.S. Application Data
(60) Provisional application No. 61/792,535, filed on Mar. 15, 2013.

(51) Int. Cl. A61M 27/00

(52) U.S. Cl. CPC A61M 27/006
USPC 604/9

(57) ABSTRACT

An intraosseus shunt can comprise a shunt body and a valve. The shunt body can be configured for insertion into bone and can have a fluid inlet, a fluid outlet, and a flow passage extending within the shunt body between the fluid inlet and the fluid outlet. The flow passage, the fluid inlet, and the fluid outlet can be configured for cerebrospinal fluid flow therethrough to cancellous bone. The valve can be attached to the shunt body and configured to control flow through the flow passage. A method of shunting biological fluid away from a hyper-pressurized biological cavity to a bone can comprise (i) creating a passage through cortical bone to provide access to cancellous bone, and (ii) positioning the shunt into the passage. The valve can control communication between the biological cavity and the cancellous bone.

### CROSS-REFERENCE TO RELATED APPLICATIONS

This application claims the priority benefit of U.S. Provisional Patent Application No. 61/792,535, filed Mar. 15, 2013, titled INTRAOSSEOUS SHUNT, the entirety of which is incorporated by reference herein.

### BACKGROUND

A variety of adverse medical conditions are associated with abnormally elevated fluid accumulation within a body cavity. These conditions include hydrocephalus, ascites, and plural effusions.

### SUMMARY

Devices and methods are disclosed herein for relieving pressure of fluid trapped in any body cavity. For example, devices and methods disclosed herein can, in some embodiments, be used to treat hydrocephalus, including, for example, communicating hydrocephalus and obstructive hydrocephalus.

Shunting cerebrospinal fluid from the lumbar cistern through a lumboperitoneal shunt can be fraught with complications, and has a published revision rate of up to 60%. Some embodiments disclosed herein may be superior to ventriculoperitoneal shunting of cerebrospinal fluid from the lumbar cistern. For example, in some embodiments, cerebral puncture can be avoided, thereby reducing the likelihood of injury to the brain. For another example, some embodiments can have a theoretical lower infection rate. As yet another example, some catheterless embodiments can be less prone to clogging than catheter-based devices, e.g. lumboperitoneal shunts. In some embodiments, a shunt can be placed in the cranium, e.g., by cerebral puncture. In some embodiments, a catheter can be attached to a shunt such that the catheter is in fluid communication with the shunt.

The subject technology is illustrated, for example, according to various aspects described below. Various examples of aspects of the subject technology are described as numbered clauses (1, 2, 3, etc.) for convenience. These are provided as examples and do not limit the subject technology. It is noted that any of the dependent clauses may be combined in any combination, and placed into a respective independent clause, e.g., Clause 1 or Clause 19. The other clauses can be presented in a similar manner.

1. An intraosseus shunt comprising:
a shunt body comprising an external surface and configured for insertion into bone;
a flow passage within the shunt body and extending between a fluid inlet and a fluid outlet, wherein the flow passage, the fluid inlet, and the fluid outlet are configured for cerebrospinal fluid flow therethrough to cancellous bone of the vertebra; and
a valve attached to the shunt body and configured to control flow through the flow passage.

2. The shunt of Clause 1, wherein the shunt body comprises a thread on the external surface

3. The shunt of Clause 2, wherein the shunt body comprises a plurality of threads on the external surface.

4. The shunt of Clause 1, wherein the shunt body further comprises a flange protruding radially outwardly from the shunt body at a location closer to an inlet end of the shunt body than an outlet and of the shunt body.

5. The shunt of Clause 4, wherein the external surface of the shunt body is substantially cylindrical, apart from the flange and the thread.

6. The shunt of Clause 1, wherein the shunt body is configured to mate with an insertion tool such the shunt body rotates with the insertion tool.

7. The shunt of Clause 1, wherein the fluid outlet comprises at least one opening through an outlet end of the shunt body.

8. The shunt of Clause 1, wherein the fluid outlet comprises a plurality of discrete openings positioned between an inlet end and an outlet and the valve body.

9. The shunt of Clause 8, wherein at least a portion of a length of the external surface comprises at least one outlet opening interspersed between thread portions.

10. The shunt of Clause 1, wherein the valve comprises a valve body, the valve body comprises an threaded external valve-body surface, the shunt body further comprises a threaded interior shunt-body surface, and the threaded external valve-body surface and the threaded interior shunt-body surface are configured for selective engagement and disengagement.

11. The shunt of Clause 10, wherein the valve body defines at least a portion of the flow passage.

12. The shunt of Clause 1, wherein the valve is a duckbill valve.

13. The shunt of Clause 1, wherein the valve comprises a plug biased against a mating shoulder.

14. The shunt of Clause 1, wherein the valve is gravity assisted.

15. The shunt of Clause 1, wherein the valve comprises a silicone membrane spanning the flow passage.

16. The shunt of Clause 1, wherein the shunt is compatible with magnetic resonance imaging.

17. The shunt of Clause 1, further comprising an antibiotic coating.

18. The shunt of Clause 1, further comprising at least one radiopaque portion.

19. A method of shunting cerebrospinal fluid from a cerebrospinal fluid cavity to a vertebra, the method comprising:
inserting a needle between posterior spinous processes of adjacent vertebra;
when the needle abuts a posterior aspect of a vertebra, inserting a dilator and cannula over the needle;
creating a passage through cortical bone, using a tool inserted through the cannula, to provide access to cancellous bone in the vertebra; and
positioning a shunt into the passage such that the cerebrospinal fluid cavity is in communication with the cancellous bone.

20. The method of Clause 19, wherein the needle is inserted between adjacent vertebra posterior spinous processes at the midline.

21. The method of Clause 19, wherein the cerebrospinal fluid cavity comprises a lumbar cistern.

22. The method of Clause 19, wherein the shunt further comprises a passage extending therethrough providing selective communication between the biological cavity and the cancellous bone controlled by a valve attached to the shunt, the valve configured to permit fluid flow and pressure above a threshold and to inhibit reflux.

23. The method of Clause 19, further comprising inserting the needle between posterior spinous processes of any two adjacent vertebra from L1 to L5.

24. The method of Clause 23, further comprising inserting the needle between posterior spinous processes of a L4 vertebra and a L5 vertebra.

25. The method of Clause 19, further comprising inserting the needle with a nerve stimulating probe.

26. The method of Clause 19, wherein the shunt is configured to be self drilling, and the passage is created by the shunt.

27. The method of Clause 19, further comprising threading the passage through cortical bone.

28. The method of Clause 27, wherein the passage is threaded using a self-tapping drilling tool.

29. The method of Clause 27, wherein the shunt is configured to be self tapping, and the passage is threaded by the shunt.

30. The method of Clause 27, wherein creating and threading the passage comprises:
(i) inserting and operating a drilling tool through the cannula to create a passage through cortical bone to provide access to cancellous bone, and
(ii), after (i), inserting and operating a tap, through the cannula, to thread the passage.

31. The method of Clause 19, wherein the shunt comprises threaded exterior surface, and positioning the shunt comprises screwing the shunt into the passage.

32. The method of Clause 31, further comprising threading the passage before screwing the shunt into the passage.

33. The method of Clause 19, wherein positioning the shunt into the passage such that the cerebrospinal fluid cavity is in communication with the cancellous bone comprises:
(i) positioning a first end portion of the shunt in the cancellous bone of the vertebra, the first end portion comprising a fluid outlet; and
(ii) positioning a second end portion of the shunt such that a fluid inlet of the second end portion is in communication with the cerebrospinal fluid cavity.

34. The method of Clause 33, wherein positioning a second end portion of the shunt such that a fluid inlet of the second end portion is in communication with the cerebrospinal fluid cavity comprises positioning the second end portion such that the second end portion, including the inlet, resides in a fluid collection.

35. The method of Clause 19, further comprising compressing the anterior dura between the vertebra in a flange protruding outwardly from an inlet end of the shunt.

36. The method of Clause 19, further comprising closing an opening in superficial dura.

37. The method of Clause 19, wherein at least one of needle insertion, passage creation, or shunt positioning is performed with image guidance.

38. A method of shunting biological fluid away from a hyper-pressurized biological cavity to a bone, the method comprising:
creating a passage through cortical bone to provide access to cancellous bone;
positioning a shunt into the passage, wherein the shunt further comprises a passage extending therethrough providing selective communication between the biological cavity and the cancellous bone controlled by a valve attached to the shunt, the valve configured to permit fluid flow and pressure above a threshold and to inhibit reflux.

39. The method of Clause 38, wherein the biological cavity is the peritoneum, the bone is an iliac crest, and fluid is shunted from the peritoneum to the iliac crest to treat ascites.

40. The method of Clause 38, wherein the biological cavity is a space between plural layers, the bone is a rib, and fluid is shunted from the space between the plural layers to the rib to treat pleural effusion.

41. The method of Clause 38, wherein the biological cavity is a cerebrospinal fluid cavity, and cerebrospinal fluid is shunted from the cerebrospinal fluid cavity to treat hydrocephalus.

42. The method of Clause 38, wherein the bone is selected from a group consisting of a vertebra, a sacrum, an iliac crest, a facial sinus, a calvarium, an orbit, a rib, a sternum, a mastoid process, a diploic space, and a tibia.

43. The method of Clause 38, further comprising threading the passage through cortical bone.

44. The method of Clause 38, wherein the passage is threaded using a self-tapping drilling tool.

45. The method of Clause 38, wherein creating and threading the passage comprises:
(i) drilling a passage through cortical bone to provide access to cancellous bone, and
(ii), after (i), tapping the passage.

46. The method of Clause 38, wherein the shunt comprises threaded exterior surface, and positioning the shunt comprises screwing the shunt into the passage.

47. The method of Clause 38, further comprising threading the passage before screwing the shunt into the passage.

48. A method of shunting biological fluid from a hyper-pressurized biological cavity to a bone, the method comprising:
positioning a first end of a tube in a vertebral body;
positioning a second end of the tube in a subarachnoid space;
wherein the tube has a length of less than 100 mm between the first end and the second end.

49. The method of Clause 48, wherein the length of the tube between the first end and the second end is less than 80 mm.

50. The method of Clause 48, wherein the length of the tube between the first end and the second end is less than 60 mm.

51. The method of Clause 48, wherein the length of the tube between the first end and the second end is less than 40 mm.

52. The method of Clause 48, wherein the length of the tube between the first end and the second end is less than 20 mm.

53. The method of Clause 48, wherein the length of the tube between the first end and the second end is less than 10 mm.

54. An intraosseus shunt comprising:
a shunt body configured for insertion into bone and having a fluid inlet, a fluid outlet, and a flow passage extending within the shunt body between the fluid inlet and the fluid outlet, wherein the flow passage, the fluid inlet, and the fluid outlet are configured for cerebrospinal fluid flow therethrough to cancellous bone;
wherein the shunt body comprises a first portion configured to extend through a pedicle of a vertebra, and second portion configured to extend into a subarachnoid space in a spinal canal, the first portion and the second portion coupled together.

55. The shunt of Clause 54, wherein when the first and second portions of the shunt are positioned to permit drainage of cerebrospinal fluid from the cavity to the cancellous bone, the first portion and the second portion extend longitudinally along respective axes at a non-zero angle relative to each other.

56. The shunt of Clause 54, further comprising a valve attached to the shunt body and configured to control flow through the flow passage.

57. The shunt of Clause 54, wherein the shunt has a length of less than 100 mm between a first end and a second end of the shunt.

58. The shunt of Clause 54, wherein the flow passage, the fluid inlet, and the fluid outlet are configured for cerebrospinal fluid flow therethrough to cancellous bone of a vertebra.

59. The shunt of Clause 54, wherein the shunt body comprises an external surface and a thread on the external surface.

60. The shunt of Clause 59, wherein the shunt body comprises a plurality of threads on the external surface.

61. The shunt of Clause 54, wherein the shunt body further comprises a flange protruding radially outwardly from the shunt body at a location closer to an inlet end of the shunt body than an outlet end of the shunt body.

62. The shunt of Clause 61, wherein the external surface of the shunt body is substantially cylindrical, apart from the flange and the thread.

63. The shunt of Clause 54, wherein the shunt body is configured to mate with an insertion tool such the shunt body rotates with the insertion tool.

64. The shunt of Clause 54, wherein the fluid outlet comprises at least one opening through an outlet end of the shunt body.

65. The shunt of Clause 54, wherein the fluid outlet comprises a plurality of openings positioned between an inlet end and an outlet end of the valve body.

66. The shunt of Clause 65, wherein at least a portion of a length of the external surface comprises at least one outlet opening interspersed between thread portions.

67. The shunt of Clause 54, wherein the valve comprises a valve body, the valve body comprises an threaded external valve-body surface, the shunt body further comprises a threaded interior shunt-body surface, and the threaded external valve-body surface and the threaded interior shunt-body surface are configured for engagement and disengagement.

68. The shunt of Clause 67, wherein the valve body defines at least a portion of the flow passage.

69. The shunt of Clause 54, wherein the valve is a duckbill valve.

70. The shunt of Clause 54, wherein the valve comprises a plug biased against a mating shoulder.

71. The shunt of Clause 54, wherein the valve is gravity assisted.

72. The shunt of Clause 54, wherein the valve comprises a silicone membrane spanning the flow passage.

73. The shunt of Clause 54, wherein the shunt is compatible with magnetic resonance imaging.

74. The shunt of Clause 54, further comprising an antibiotic coating.

75. The shunt of Clause 54, further comprising at least one radiopaque portion.

76. A method of shunting cerebrospinal fluid from a cerebrospinal fluid cavity to a vertebra, the method comprising:
creating a passage through cortical bone, and into cancellous bone, of the vertebra; and
positioning a shunt such that the cerebrospinal fluid cavity is in communication with the cancellous bone.

77. The method of Clause 76, wherein positioning the shunt comprises inserting a first portion of the shunt through a pedicle of the vertebra and through the passage, and inserting a second portion of the shunt into the cerebrospinal fluid cavity.

78. The method of Clause 76, wherein the cerebrospinal fluid cavity comprises a subarachnoid space from L1 to L5.

79. The method of Clause 76, wherein, when the first and second portions of the shunt are positioned to permit drainage of cerebrospinal fluid from the cavity to the cancellous bone, the first portion and the second portion extend longitudinally along respective axes at a non-zero angle relative to each other.

80. The method of Clause 76, wherein positioning the shunt comprises coupling the first portion and the second portion together.

81. The method of Clause 76, further comprising inserting a needle between posterior spinous processes of adjacent vertebra and, when the needle abuts a posterior aspect of a vertebra, inserting a dilator and cannula over the needle.

82. The method of Clause 76, wherein the cerebrospinal fluid cavity comprises a lumbar cistern.

83. The method of Clause 76, wherein the shunt has a passage extending therethrough providing regulated communication between the biological cavity and the cancellous bone controlled by a valve attached to the shunt, the valve configured to permit fluid flow and pressure above a threshold and to inhibit reflux.

84. The method of Clause 76, further comprising inserting a needle with a nerve stimulating probe.

85. The method of Clause 76, wherein the shunt is configured to be self-drilling, and the passage is created by the shunt.

86. The method of Clause 76, further comprising threading the passage through cortical bone.

87. The method of Clause 86, wherein the passage is threaded using a self-tapping drilling tool.

88. The method of Clause 86, wherein the shunt is configured to be self-tapping, and the passage is threaded by the shunt.

89. The method of Clause 86, wherein creating and threading the passage comprises:
(i) inserting and operating a drilling tool through a cannula to create a passage through cortical bone to provide access to cancellous bone, and
(ii), after (i), inserting and operating a tap, through the cannula, to thread the passage.

90. The method of Clause 76, wherein the shunt comprises a threaded exterior surface, and positioning the shunt comprises screwing the shunt into the passage.

91. The method of Clause 90, further comprising threading the passage before screwing the shunt into the passage.

92. The method of Clause 76, wherein positioning the shunt such that the cerebrospinal fluid cavity is in communication with the cancellous bone comprises:
(i) positioning a first end portion of the shunt in the cancellous bone of the vertebra, the first end portion comprising a fluid outlet; and
(ii) positioning a second end portion of the shunt such that a fluid inlet of the second end portion is in communication with the cerebrospinal fluid cavity.

93. The method of Clause 92, wherein positioning a second end portion of the shunt such that a fluid inlet of the second end portion is in communication with the cerebrospinal fluid cavity comprises positioning the second end portion such that the second end portion, including the inlet, resides in a fluid collection.

94. The method of Clause 76, further comprising compressing the dura between the vertebra and a flange protruding outwardly from an inlet end of the shunt.

95. The method of Clause 76, further comprising closing an opening in superficial dura.

96. The method of Clause 76, wherein at least one of needle insertion, passage creation, or shunt positioning is performed with image guidance.

97. A method of shunting biological fluid away from a pressurized biological cavity to a bone, the method comprising:
creating a passage through cortical bone to provide access to cancellous bone;
positioning a shunt into the passage, wherein the shunt comprises a lumen extending therethrough providing communication between the biological cavity and the cancellous bone controlled by a valve coupled to the shunt, the valve configured to permit fluid flow and pressure above a threshold and to inhibit reflux.

98. The method of Clause 97, wherein the biological cavity is the peritoneum, the bone is an iliac crest, and fluid is shunted from the peritoneum to the iliac crest to treat ascites.

99. The method of Clause 97, wherein the biological cavity is a space between plural layers, the bone is a rib, and fluid is shunted from the space between the plural layers to the rib to treat pleural effusion.

100. The method of Clause 97, wherein the biological cavity is a cerebrospinal fluid cavity, and cerebrospinal fluid is shunted from the cerebrospinal fluid cavity to treat hydrocephalus.

101. The method of Clause 97, wherein the bone is selected from a group consisting of a cranium, a vertebra, a sacrum, an iliac crest, a facial sinus, a calvarium, an orbit, a rib, a sternum, a mastoid process, a diploic space, and a tibia.

102. The method of Clause 97, further comprising threading the passage through cortical bone.

103. The method of Clause 97, wherein the passage is threaded using a self-tapping drilling tool.

104. The method of Clause 97, wherein creating and threading the passage comprises:
(i) drilling a passage through cortical bone to provide access to cancellous bone, and
(ii), after (i), tapping the passage.

105. The method of Clause 97, wherein the shunt comprises threaded exterior surface, and positioning the shunt comprises screwing the shunt into the passage.

106. The method of Clause 105, further comprising threading the passage before screwing the shunt into the passage.

107. A method of shunting biological fluid from a pressurized biological cavity to a bone, the method comprising:
positioning a first end of a tube in a vertebral body;
positioning a second end of the tube in a subarachnoid space;
wherein the tube has a length of less than 100 mm between the first end and the second end.

108. The method of Clause 107, wherein the length of the tube between the first end and the second end is less than 80 mm.

109. The method of Clause 107, wherein the length of the tube between the first end and the second end is less than 60 mm.

110. The method of Clause 107, wherein the length of the tube between the first end and the second end is less than 40 mm.

111. The method of Clause 107, wherein the length of the tube between the first end and the second end is less than 20 mm.

112. The method of Clause 107, wherein the length of the tube between the first end and the second end is less than 10 mm.

113. An intraosseus shunt comprising:
a shunt body configured for insertion into bone and having a fluid inlet, a fluid outlet, and a flow passage extending within the shunt body between the fluid inlet and the fluid outlet, wherein the flow passage, the fluid inlet, and the fluid outlet are configured for cerebrospinal fluid flow therethrough to cancellous bone; and
a valve attached to the shunt body and configured to control flow through the flow passage.

Additional features and advantages of the subject technology will be set forth in the description below, and in part will be apparent from the description, or may be learned by practice of the subject technology. The advantages of the subject technology will be realized and attained by the structure particularly pointed out in the written description and claims hereof as well as the appended drawings.

It is to be understood that both the foregoing general description and the following detailed description are exemplary and explanatory and are intended to provide further explanation of the subject technology as claimed.

### BRIEF DESCRIPTION OF THE DRAWINGS

The accompanying drawings, which are included to provide further understanding of the subject technology and are incorporated in and constitute a part of this description, illustrate aspects of the subject technology and, together with the specification, serve to explain principles of the subject technology.

FIG. 1 is a sagittal cross-sectional view of a lower spinal column with an intraosseous shunt, according to an embodiment, placed at an L5 vertebra.

FIG. 2 is a side view of a shunt, according to an embodiment.

FIG. 3 is a cross-sectional side view of a shunt comprising a gravity-assisted valve, according to an embodiment.

FIG. 4 is a cross-sectional side view of a shunt comprising a spring-based valve, according to an embodiment.

FIG. 5 is a cross-sectional side view of a shunt comprising a diaphragm valve, according to an embodiment.

FIG. 6 is a cross-sectional side view of a shunt comprising a duckbill valve, according to an embodiment.

FIG. 7 is a sagittal cross-sectional view of a lower spinal column with an intraosseous shunt, according to an embodiment, placed at an L3 vertebra.

FIG. 8 is a transverse cross-sectional view of a vertebra and an intraosseous shunt, according to an embodiment.

FIG. 9 is a sagittal cross-sectional view of a cranium and an intraosseous shunt, according to an embodiment.

FIG. 10 is a sagittal cross-sectional view of a cranium and an intraosseous shunt, according to an embodiment.

FIG. 11 is a sagittal cross-sectional view of a cranium and an intraosseous shunt, according to an embodiment.

FIG. 12 is a sagittal cross-sectional view of a cranium and an intraosseous shunt, according to an embodiment.

### DETAILED DESCRIPTION

In the following detailed description, specific details are set forth to provide an understanding of the subject technology. It will be apparent, however, to one ordinarily skilled in the art that the subject technology may be practiced without some of these specific details. In other instances, well-known structures and techniques have not been shown in detail so as not to obscure the subject technology.

FIG. 1 illustrates an exemplifying use of an intraosseous shunt, according to an embodiment. More specifically, FIG. 1 is a sagittal cross-sectional view of a lower spinal column with an intraosseous shunt 100 inserted into a vertebra to treat hydrocephalus. Certain embodiments are described with reference to treatment of hydrocephalus. However, the present invention is not so limited. Various embodiments of the devices and methods can be used to relieve pressure of fluid trapped in any body cavity. For example, various devices and methods disclosed can be used to treat hydrocephalus, ascites, pleural effusions, and other conditions.

FIGS. 2-6 schematically illustrate various aspects of shunts 100, according to some embodiments of the subject technology. FIG. 2 is a side view of a shunt, illustrating various aspects of shunts according to some embodiments. FIGS. 3-6 are cross-sectional side views of shunts 100 and show, among other features, various types of valves 120 configured to regulate flow through shunts according to some embodiments.

As illustrated in FIGS. 2 and 3, the shunt 100 can comprise a shunt body 102, a fluid inlet 104, a fluid outlet 106, and a flow passage 108 connecting the fluid inlet and the fluid outlet. The shunt body 102 can comprise one or more components. In some embodiments, the shunt body can be formed of a single unitary component. In some embodiments, the shunt body can be formed of multiple components assembled together. An external surface 110 of the shunt body 102 can be configured for insertion into bone. For example, the external surface 110 can comprise one or more threads 112 to assist retention of the shunt 100 in bone. The shunt 100 can comprise a stop member 114, in some embodiments. In some embodiments, the shunt 100 can be configured to facilitate manipulation of the shunt by one or more tools.

The external surface 110 of the shunt body 102 can be generally or substantially cylindrical to permit, or facilitate, rotation of the shunt body 102 in an opening in bone. In embodiments wherein the shunt 100 comprises one or more threads 112, the threads can be contiguous or discontinuous. In some embodiments, the shunt body 102 can be configured for insertion into bone without threads. For example, the external surface 110 can be smooth in some embodiments. As another example, the external surface 110 can comprise teeth, hooks, barbs, serrations, or other projections configured to retain the shunt 100 in bone.

The external surface 110 can comprise a tool engagement portion 116, for example, as illustrated in FIG. 2. Additionally or alternatively, the shunt body 102 can comprise a tool engagement portion 118 on an interior surface, for example, as illustrated in FIG. 3. The tool engagement portions 116, 118 can be shaped for cooperative engagement with a tool to facilitate manipulation of the shunt body 102 by a user. For example, the tool engagement portions can comprise a plurality of surfaces arranged as a pentagon, pentalobe, hexagon, octagon, or other arrangement. The tool engagement portions can be configured to mate with an insertion tool facilitate advancement, retraction, rotation, or other motion of the shunt body 102.

The shunt body 102 can comprise a stop member 114 in some embodiments, and can omit the stop member 114 in others. The stop member 114 can protrude radially outwardly from the shunt body 102. The stop member 114 can be located along the shunt body closer to an inlet end of the shunt body than an outlet end of the shunt body. In various embodiments, the stop member 114 can be spaced from one or both ends of the shunt. FIG. 2 illustrates a stop member 114 comprising a flange that surrounds the shunt body 102. In various embodiments, the stop member 114 can comprise a flange that partially or completely surrounds the shunt body 102. The stop member 114 can comprise a shoulder in some embodiments. In some embodiments, the stop member 114 can be flat, concave, or convex. In some embodiments, the stop member 114 can have a uniform or substantially uniform radial dimension measured from (i) the shunt body 102, a central axis of the shunt 100 (if present), or both, to (ii) a radially outermost periphery of the stop member. In some embodiments, the stop member 114 can have a radial dimension measured from (i) the shunt body 102, a central axis of the shunt 100 (if present), or both, to (ii) a radially outermost periphery of the stop member that varies about the shunt body 102. In some embodiments, the stop member 114, portion(s) of the shunt body 102 positioned to pass through cortical bone, or both can be configured, e.g., sized and shaped, to provide a seal against the adjacent tissue, when the shunt is implanted, such that passage of fluid between the adjacent tissue and the stop member 114, portion(s) of the shunt body 102, or both is prevented or inhibited.

The fluid outlet 106 can comprise a plurality of openings, for example, as illustrated in FIG. 2. Although FIG. 2 shows six outlet openings, the fluid outlet 106 can comprise any number of openings. In some embodiments, the openings of the fluid outlet 106 can be spaced apart from each other and can be positioned along all or a portion of the length of the shunt body 102 between the shunt body's proximal and distal ends. The openings in the fluid outlet 106 can be interspersed with portions of the threads 112, for example, as illustrated in FIG. 2. In some embodiments, the fluid outlet 106 can comprise a single opening, for example at or near an end, e.g., a distal end, of the shunt body 102. The fluid outlet 106 can be sufficiently large to permit adequate absorption of the bodily fluid, e.g., cerebrospinal fluid, by cancellous bone at or near the fluid outlet 106. In some embodiments, the fluid outlet 106 can be spaced from the fluid inlet 104 by a section of the shunt body 102 that has no holes between the passage 108 and an exterior of the shunt 100. For example, the section having no such holes can have a length sufficient to span a subdural space, a total thickness of a layer of cortical bone, or both, at a location where the shunt is to pass through one or both of them. In some embodiments, the section having no such holes can have a length greater than is sufficient to span a subdural space, a total thickness of a layer of cortical bone, or both, at a location where the shunt is to pass through one or both of them.

As illustrated in FIG. 3, for example, the fluid inlet 104 can comprise a single opening. However, the fluid inlet 104 can comprise a plurality of openings in some embodiments (see, e.g., FIG. 7). The fluid inlet 104 can be located at or near an end, e.g., a proximal end, of the shunt body 102.

The fluid inlet 104, fluid outlet 106, and the flow passage 108 can be configured for flow of cerebrospinal fluid, or other body fluids, therethrough to cancellous bone, trabecular bone, bone marrow, or a medullary cavity. The viscosity of the body fluid to be transmitted through the shunt 100 and the desired flow rate of the body fluid through the shunt influence the selection of dimensions for the fluid inlet 104, the fluid outlet 106, and the flow passage 108. The number and size of openings of the fluid inlet and fluid outlet and the cross-sectional area and length of the flow passage 108 can be selected based on the viscosity of the transmitted fluid and the desired flow rate.

The openings of the fluid inlet and the fluid outlet can be configured in various manners. For example, the openings can be circular, ovoid, polygonal, or other shapes. In some embodiments, the openings can be configured as slots.

The movement of bodily fluid through the flow passage 108 can be regulated by one or more valves 120, such as, for example, those illustrated in FIGS. 3-6. A valve 120 can comprise a valve body 122 configured for engagement with and disengagement from the shunt body 102. For example, as illustrated in FIGS. 2 and 3, the shunt body 102 can comprise an internal thread and the valve body 122 can comprise an external thread 124 configured to mate with the internal thread 126 of the shunt body. Additionally or alternatively, the valve body 122 can be configured to snap into the shunt body 102. In some embodiments, the ability to engage and disengage the shunt body 102 and the valve body 122 can advantageously permit exchange of valves 120 within the shunt body without removing the shunt body from a bone wherein the shunt body resides, e.g., in the event that the valve comes clogged or fails.

As illustrated in FIGS. 3-6, for example, the valve body 122 can form a portion of the flow passage 108 connecting the fluid inlet 104 and the fluid outlet 106, and the valve can regulate the flow of fluid by partially or totally obstructing flow through the valve body.

FIG. 3 is a cross-sectional side view of a shunt comprising a gravity-assisted valve 120. The gravity-assisted valve of FIG. 3 regulates the flow of fluid based on the inlet fluid pressure in the orientation of the valve relative to gravity. For example, when the gravity-assisted valve is oriented horizontally, as shown in FIG. 3 for example, a lesser amount of inlet fluid pressure is required to displace the balls 128 such that fluid flows through the passage 108, than when the valve is oriented vertically. Exemplifying gravity-assisted valves include the PROGAV® valves sold by B. Braun Melsungen AG.

FIG. 4 is a cross-sectional side view of a shunt comprising a spring-biased valve, according to an embodiment. The valve 120 of FIG. 4 comprises a ball 128, a frustoconical surface 130, and a spring 132. The spring is positioned between a fluted insert 134 and the ball 128, and biases the ball toward the frustoconical surface. The insert 134 is fixed or substantially fixed against longitudinal movement within the valve body 122. The flutes of the insert 134 permit fluid to flow between the insert and the valve body.

FIG. 5 is a cross-sectional side view of a shunt comprising a diaphragm valve, according to an embodiment. The valve 120 of FIG. 5 comprises a diaphragm 136 that spans the flow passage 108. The diaphragm 136 can be configured to flex in response to inlet fluid pressure above a threshold to permit flow through the valve 120. The dimensions of the diaphragm depend upon the material that forms the diaphragm and the size of the flow passage. In some embodiments, the diaphragm can comprise silicone or other bio-acceptable material. Exemplifying diaphragm-regulated valves include those sold by Integra NeuroSciences (Plainsboro, N.J.) under the EQUI-FLOW™ mark.

FIG. 6 is a cross-sectional side view of a shunt comprising a duckbill valve, according to an embodiment. The duckbill valve 120, as illustrated in FIG. 6 for example, can comprise one or more flaps 138 that are biased together and, in the absence of external forces, close the flow passage 108. The flaps 138 are shown in FIG. 6 in a partially open state, such as would occur, for example, when an inlet fluid pressure exceeds a threshold pressure. Although the term “duckbill” has been used to describe the valve illustrated in FIG. 6, the duckbill valve can comprise any number of flaps 138. For example, in some embodiments, a duckbill valve can be a tricuspid valve.

FIGS. 3-6 illustrate an exemplifying variety of valve types. However, the subject technology is not limited to any particular type of valve. In some embodiments, the valve 120 can comprise a flap, spring, ball, or other types of valves.

The valve 120 can be configured to open with an inlet fluid pressure exceeds a threshold pressure. Threshold pressure can be selected based on acceptable pressure ranges for fluid within a body cavity for treatment. Closure of the valve can reduce or prevent reflux of fluid from the medullary cavity toward the fluid cavity, e.g., a subarachnoid space.

FIG. 7 illustrates a shunt 100 according to an embodiment. As illustrated in FIG. 7, for example, the shunt 100 can have a length, total or from the stop member 114 (if present), that is at least sufficient to extend through the lumbar cistern with a distal end positioned in the medullary cavity of a vertebra. As also illustrated in FIG. 7, for example, a proximal portion end of the shunt 100 can extend through the superficial dura (e.g., the thecal sac) such that the proximal end is located in the subfascial or subcutaneous space, in some embodiments. In some embodiments, the shunt body 102 can be configured to reside in an obstructed opening of the superficial dura made during placement of the shunt. In some embodiments, the shunt can seal the opening of the superficial dura (e.g., the thecal sac) made during placement of the shunt.

In some embodiments, configuration of the shunt body 102 to extend across the entire lumbar cistern (e.g., across a width as viewed in a sagittal plane) can inhibit, or prevent, nerve roots that float in the cerebrospinal fluid of the lumbar cistern or other tissues from obstructing the inlet 104. In some embodiments, the shunt 100 can have a length, total or from the stop member 114 (if present), that is at least sufficient to position the proximal end 152 in lumbar cistern such that no portion of the shunt resides within the subfascial or subcutaneous space.

A portion of the shunt body 102 can be configured to be positioned in the lumbar cistern. The fluid inlet 104 of the shunt 100 can comprise a plurality of openings, for example, as illustrated in FIG. 7, located along the shunt body 102 such that the inlet openings are positioned in lumbar cistern when the shunt 100 is installed in a vertebra.

As illustrated in FIG. 7, the stop member 114 can be positioned along the shunt body 102 closer to a distal end 150 than to a proximal end 152.

In some embodiments, the shunt 100 can comprise an interrogation member 154, for example, as illustrated in FIG. 7. The interrogation member 154 can provide access to the shunt for interrogation, for example by insertion of a needle to the interrogation member to a reservoir in communication with the flow passage 108 within the shunt 100. The inclusion of an interrogation member 154 can be particularly advantageous in embodiments wherein the shunt 100 traverses the lumbar cistern and a proximal end 152 is positioned in the subfascial or subcutaneous space. In some embodiments, the interrogation member 154 can be formed of silicone can provide a reservoir in direct or indirect communication with the flow passage 108, the lumbar cistern, or both. In some embodiments, interrogation member 154 is configured as a cap attached to the shunt body 102 so as to cover an opening, in the shunt body 102, in communication with the flow passage 108.

In some embodiments, the shunt 100 can be configured to be compatible with magnetic resonance imaging. For example, the shunt 100 can be formed of materials compatible with magnetic resonance imaging. In some embodiments, the shunt 100 can comprise at least one radiopaque portion. For example, the shunt body 102 can be formed of a radiopaque material. As another example, the shunt 100 can carry a radiopaque marker.

In some embodiments, the shunt 100 can include one or more coatings. For example, shunt 100 can comprise an antibiotic coating.

In some embodiments, fluid can be shunted from a hyper-pressurized biological cavity to a bone. A passage can be created through the cortex of the bone to provide access to the medullary cavity of the bone. A shunt 100 can be positioned in the passage to provide regulated fluid communication between the biological cavity and the medullary cavity controlled by a valve 120 in the shunt. In some embodiments, the passage through cortical bone can be threaded before insertion of the shunt into the bone.

In various embodiments, fluid can be shunted into a bone selected from the group consisting of a cranium, a vertebra, a sacrum, an iliac crest, a facial sinus, a calvarium, an orbit, a rib, a sternum, a mastoid process, a diploic space, and a tibia.

In some embodiments, fluid is shunted from the peritoneum to an iliac crest to treat ascites. In some embodiments, fluid is shunted from a space between the plural layers to a rib to treat pleural effusions. In some embodiments, hydrocephalus can be treated by shunting cerebrospinal fluid into a vertebra, a cranium, or both.

Exemplifying methods of shunting cerebrospinal fluid to a vertebra for treating hydrocephalus are described with reference to FIG. 1. Although specific reference is made to cerebrospinal fluid and vertebra, for example, the described methods can be applied to bones, fluids, conditions, etc. other than those specifically referenced. For example, cerebrospinal fluid can be shunted from any cerebrospinal fluid cavity into any bone. The embodiments disclosed herein may be particularly advantageous when shunting cerebrospinal fluid from a cerebrospinal fluid cavity into a bone adjacent to the cerebrospinal fluid cavity. In some embodiments, the fluid path is advantageously short. For example, the distance between the fluid inlet and the fluid outlet can be less than 100 mm, 90 mm, 80 mm, 70 mm, 60 mm, 50 mm, 40 mm, 30 mm, 25 mm, 20 mm, 15 mm, 10 mm, 5 mm, or 2 mm in some embodiments.

A needle can be inserted between posterior spinous processes 186 of adjacent vertebra and directed to a target vertebral body. Any vertebral body can be targeted as the shunt 100, according to various embodiments, can be placed in any vertebra. For example, in some embodiments, the shunt 100 can be placed in a L3, L4, or L5 vertebra. In some embodiments, multiple shunts 100 can be placed in the same vertebra, multiple vertebrae, or both. The needle can be inserted as if performing a lumbar puncture, and can be inserted at or near the midline. In some embodiments the needle is inserted between posterior spinous processes of two adjacent vertebra, e.g., of two adjacent vertebra from L1 to L5. For example, in some embodiments, the needle is inserted between posterior spinous processes of a L4 vertebra and a L5 vertebra to place a shunt 100 in the L4 vertebra or the L5 vertebra. As a further example, in some embodiments, the needle is inserted between posterior spinous processes of a L3 vertebra and a L4 vertebra to place a shunt 100 in the L3 vertebra or the L4 vertebra. As yet another example, in some embodiments, the needle is inserted between posterior spinous processes of a L2 vertebra and a L3 vertebra to place a shunt 100 in the L2 vertebra or the L3 vertebra. Inserting the needle with a nerve stimulating probe can be advantageous in some embodiments.

When the needle abuts a posterior aspect of a vertebra, a dilator and cannula can be inserted over the needle. The needle can be removed after insertion of the dilator and cannula. In some embodiments, a guide wire can be placed through the needle, the needle can be removed, and the dilator and cannula can be inserted over the guide wire. Access to the bone can be obtained in these and various other ways in some embodiments. A tool can be inserted and manipulated through the cannula to create a passage 140 through the cortex, cortical bone, or compact bone 142 of the vertebra to provide access to the cancellous bone, trabecular bone, marrow, or medullary cavity 144 of the vertebra.

In some embodiments wherein a shunt 100 comprising external threads 112 is to be placed in the bone, the passage 140 through the cortex can be threaded in preparation for insertion of the shunt 100. For example, the passage 140 can be created and threaded in a single operation using a self-tapping drilling tool. Alternatively, the passage 140 can be created by inserting and operating a drilling tool through the cannula, removing the drilling tool, and thereafter inserting operating a tap, through the cannula, to thread the passage 140. The passage 140 need not be threaded before insertion of the shunt 100 if the shunt comprises external threads 112. In some embodiments, the shunted 100 can be self-drilling, self-tapping, or both.

After creation of the passage 140, the shunt 100 can be inserted into the bone such that the fluid outlet 106 (FIGS. 2-6) is adjacent the cancellous bone, trabecular bone, marrow, or medullary cavity 144 of the vertebra. In embodiments wherein the shunt 100 comprises external threads 122, positioning the shunt 100 in the passage 140 can comprise rotating the shunt using a tool through the cannula. The fluid inlet 106 (FIGS. 2-6) can be positioned in fluid communication with the fluid cavity. For example, in various embodiments, the fluid inlet can be inserted into the bone, flush with an exterior surface of the bone, or protrude from the bone. In some embodiments, the fluid cavity can be a subarachnoid space or lumbar cistern.

In embodiments wherein the shunt 100 comprises a valve body 122 that is able to engage with and disengage from the shunt body 102, the valve body 122 can be inserted into the patient's body together with the shunt body 102, or can be inserted subsequent to placement of the shunt body in the body. For example, the valve body 122 can be screwed, snapped, or otherwise engaged with the shunt body 102 after the shunt body has been positioned in the bone.

In embodiments wherein the shunt 100 comprises a stop member 114, the anterior dura 146 (e.g., the thecal sac) can be compressed between the vertebra and the stop member 114 when the shunt is placed in the body or by subsequent action.

After placement of the shunt 100 in the vertebra, an access opening in the superficial dura 148 can be closed. For example, a one or more stitches can be placed, a vascular closure device can be used or both, if desirable or necessary. Closure devices that can be used include, for example, the STARCLOSE and PERCLOSE devices sold by Abbot Vascular.

In embodiments wherein the shunt 100 is configured in position to traverse the lumbar cistern and extend through the superficial dura, the shunt 100 can obstruct or seal the opening in the superficial dura was made for placement of the shunt. In some such embodiments, closure of the superficial dura the omitted as unnecessary.

In embodiments wherein the shunt 100 comprises an interrogation member 154, some methods can comprise tapping the interrogation member 154 with a needle to draw fluid from a reservoir therein.

In some embodiments, the shunt 100 can be placed in bone by an open surgery using techniques similar to those described above.

In some embodiments, any or all of the methods described herein can be performed using medical imaging techniques. For example, some procedures can be performed using magnetic resonance imaging. In some embodiments, one or more of needle insertion, creation of a passage in bone, shunt positioning, or closure can be performed under imaging. In some embodiments, a shunt can be placed percutaneously aided use of one or more medical imaging modalities, such as, for example, fluoroscopic guidance.

In some instances, e.g., to reduce a possibility of damaging nerves (e.g., nerve roots 184, FIG. 8) within the thecal sac, it may be desirable not to manipulate the nerves during placement of the shunt, e.g., not to implant a shunt by moving the shunt's outlet portion through the thecal sac to place the fluid outlet at a drainage site in bone. FIG. 8 illustrates an exemplifying use of an intraosseous shunt, according to some aspects of the subject technology, having a fluid outlet 106 placed at a drainage site in bone without passing the outlet portion of the shunt through the thecal sac. More specifically, FIG. 8 shows a transverse cross-sectional view of a vertebra with an intraosseous shunt 100 inserted into a pedicle of the vertebra to treat hydrocephalus. In FIG. 8, as elsewhere herein, the same reference numerals are used to designate features that are the same or similar, except as otherwise noted. Thus, some description of such features is omitted in some instances to avoid repetition.

As illustrated in FIG. 8, for example, the shunt 100 can comprise a shunt body 102, a fluid inlet 104, a fluid outlet 106, and a flow passage 108 connecting the fluid inlet and the fluid outlet. The shunt body 102 can comprise one or more components. For example, as illustrated in FIG. 8, the shunt body 102 can comprise a first shunt body member 156 and a second shunt body member 158. The flow passage 108 can extend through both, or all, shunt body members 156, 158, for example, as illustrated in FIG. 8, or can extend through only one shunt body member. The first shunt body member 156 can comprise the fluid outlet 106 for allowing fluid to pass from the flow passage 108 to a drainage site. The second shunt body member 158 can comprise the fluid inlet 104 for allowing fluid pass from a fluid collection into the flow passage 108.

An external surface 110 of the first shunt body member 156 can be configured for insertion into bone. For example, the external surface 110 can comprise one or more threads 112 to assist retention of the shunt 100 in bone. The one or more threads 112 can extend proximal to, distal to, and/or along the opening(s) of the fluid outlet 106. In some embodiments, the first shunt body member 156 can have a distal end that is self-drilling, self-tapping, or both.

The shunt 100 can comprise a stop member 114 (see, e.g., FIGS. 1-7), in some embodiments.

In some embodiments, the shunt 100 can be configured to facilitate manipulation of the shunt by one or more tools (see, e.g., FIGS. 1-7).

The external surface 110 of the first shunt body member 156 can be generally or substantially cylindrical to permit, or facilitate, rotation of the first shunt body member 156 in an opening in bone. In embodiments wherein the first shunt body member 156 comprises one or more threads 112, the threads can be contiguous or discontinuous. In some embodiments, the first shunt body member 156 can be configured for insertion into bone without threads. For example, the external surface 110 can be smooth in some embodiments. As another example, the external surface 110 can comprise teeth, hooks, barbs, serrations, or other projections configured to retain the shunt 100 in bone.

The second shunt body member 158 can comprise a proximal end 160 and a distal end 162. The fluid inlet 104 can be located at or near an end, e.g., a distal end 162, of the second shunt body member 158. As illustrated in FIG. 8, for example, the fluid inlet 104 can comprise a single opening. However, the fluid inlet 104 can comprise a plurality of openings in some embodiments.

The proximal end 160 of the second shunt body member 158 can be configured for attachment to, engagement with, and/or coupling with the first shunt body member 156. In some embodiments, for example, as illustrated in FIG. 8, the proximal end 160 of the second shunt body member 158 can be configured to be placed over a proximal end 164 of the first shunt body member 156. In some embodiments, the proximal end 160 of the second shunt body member 158 can be configured to be placed with the proximal end 164 of the first shunt body member 156. In some embodiments, the proximal end 160 of the second shunt body member 158 and the proximal end 164 of the first shunt body member 156 can have complementary shapes for mating attachment, engagement, and/or coupling to each other, e.g, by interference fit, threaded engagement, or otherwise, or a combination thereof.

In some embodiments, the second shunt body member 158 can comprise a tubular portion, e.g., extending proximally from the distal end 162. In some embodiments, the tubular portion can extend from the distal end 162 to a portion configured to connect to another shunt body member, e.g., the first shunt body member 156.

The second shunt body member 158 can have a length between the proximal end 160 and the distal end 162 sufficient to extend from the proximal end 164 of the first shunt body member 156 into a fluid cavity, e.g., a lumbar cistern (FIG. 8) or a subarachnoid space (FIGS. 10-12). For example, the length between the proximal end 160 and the distal end 162 can be less than 100 mm, 90 mm, 80 mm, 70 mm, 60 mm, 50 mm, 40 mm, 30 mm, 25 mm, 20 mm, 15 mm, 10 mm, 5 mm, or 2 mm in some embodiments.

The second shunt body member 158 can extend through the superficial dura (e.g., the thecal sac) such that the distal end 162 is located in the subarachnoid space, in some embodiments. In some embodiments, the second shunt body member 158 can be configured to reside in an obstructed opening of the superficial dura made during placement of the shunt 100. In some embodiments, the second shunt body member 158 can seal the opening of the superficial dura (e.g., the thecal sac) made during placement of the shunt.

In some embodiments, the second shunt body member 158 illustrated in FIG. 8 can comprise multiple components and/or portions comprising different materials. In some embodiments, all or a portion of the second shunt body member 158, e.g., a distal portion extending into the fluid cavity, can comprise biocompatible metal(s), polymer(s), or other materials, or combinations thereof. In some embodiments, all or a portion of the second shunt body member 158 can comprise stainless steel, titanium, or other metal(s), or combinations thereof. In some embodiments, all or a portion of the second shunt body member 158 can comprise silicone, plastic, or other material(s), or combinations thereof. In some embodiments, a portion extending into the fluid cavity can be flexible.

As discussed in greater detail above, the fluid inlet 104, fluid outlet 106, and the flow passage 108 can be configured for flow of cerebrospinal fluid, or other body fluids, therethrough to cancellous bone, trabecular bone, bone marrow, or a medullary cavity. Also as discussed in greater detail above, the movement of bodily fluid through the flow passage 108 can be regulated by one or more valves 120, such as, for example, those illustrated (e.g., in FIGS. 3-6) and/or otherwise disclosed herein. The valve can form a portion of the flow passage 108 connecting the fluid inlet 104 and the fluid outlet 106, and the valve can regulate the flow of fluid by partially or totally obstructing flow through the valve body. The valve can be positioned partially or wholly within the shunt body 102. In some embodiments, the valve can be positioned partially or wholly within one or more components, e.g., the first shunt body member 156 or the second shunt body member 158, of the shunt body. Additionally or alternatively, the valve can be positioned between other components of the shunt body, in some embodiments. In some embodiments, the valve can form a portion of the shunt body.

In some embodiments, the shunt 100 can comprise an interrogation member 154. The interrogation member can be attached to or formed as part of one or more components of the shunt body 102. For example, the interrogation member can be formed as part of the second shunt body member 158, e.g., proximate to a proximal end of the second shunt body member, for example, as illustrated in FIG. 8.

The shunt 100 illustrated in FIG. 8 can be minimally invasively implanted in a patient's body. The first shunt body member 156 can be positioned through a pedicle 166 such that the fluid outlet 106 is adjacent the cancellous bone, trabecular bone, marrow, or medullary cavity 144 of the vertebra. In some embodiments, techniques such as those discussed above can be used place the shunt illustrated in FIG. 8 and similar shunts.

After obtaining access to a posterior aspect of a vertebra, a tool can be inserted and manipulated to create a passage through the pedicle 166 to provide access to the cancellous bone, trabecular bone, marrow, or medullary cavity 144 of the vertebra.

In some embodiments wherein a shunt 100 comprising external threads 112 is to be placed in the bone, the pedicle 166 can be threaded in preparation for insertion of the shunt 100. For example, a passage 140 through the pedicle 166 can be created and threaded in a single operation using a self-tapping drilling tool. Alternatively, the passage can be created by operating a drilling tool, removing the drilling tool, and thereafter operating a tap, through the cannula, to thread the passage 140. The passage 140 need not be threaded before insertion of the shunt 100 if the shunt comprises external threads 112. In some embodiments, the shunted 100 can be self-drilling, self-tapping, or both. After creation of the passage 140, the shunt 100 can be inserted into the pedicle 166 such that the fluid outlet 106 is adjacent the cancellous bone, trabecular bone, marrow, or medullary cavity 144, for example, as discussed above.

The fluid inlet 104 can be positioned in fluid communication with the fluid cavity. For example, in various embodiments, the fluid inlet can be inserted through the lamina 168, and the dura 146, e.g., using a Tuohy needle. In some embodiments, the distal end 162 of the second shunt body member 158 can extend into the lumbar cistern 170.

In embodiments wherein the shunt 100 comprises a valve body 122 that is engagable with and disengagable from the shunt body 102 (e.g., the first shunt body member 156) the valve body 122 can be inserted into the patient's body together with the shunt body 102, or can be inserted subsequent to placement of the shunt body in the body. For example, the valve body 122 can be screwed, snapped, or otherwise engaged with the shunt body 102 (e.g., the first shunt body member 156) after the shunt body has been positioned in the bone.

In embodiments wherein the shunt 100 comprises multiple shunt body components (e.g., the first shunt body member 156 and the second shunt body member 158), shunt body components can be attached, engaged, and/or coupled to each other before or after placement of some, all, or none of the shunt body components. For example, in some embodiments, the first shunt body member 156 and the second shunt body member 158 can be coupled to each other after each of them has been independently placed in the vertebra.

After placement of the shunt 100 in the vertebra, any access opening(s) can be closed, if necessary or desired.

In embodiments wherein the shunt 100 comprises an interrogation member 154, some methods can comprise tapping the interrogation member 154 with a needle to draw fluid from a reservoir therein.

In some embodiments, the shunt 100 can be placed in bone by an open surgery using techniques similar to those described above.

In some embodiments, any or all of the methods described herein can be performed using medical imaging techniques. For example, some procedures can be performed using magnetic resonance imaging or fluoroscopic guidance. In some embodiments, one or more of needle insertion, creation of a passage in bone, shunt positioning, or closure can be performed under imaging.

FIGS. 9-12 illustrate exemplifying uses of an intraosseous shunt, according to some embodiments. More specifically, FIGS. 9-12 are a sagittal cross-sectional views of craniums and intraosseous shunts 100 inserted through the cranial bone 180 into either the subarachnoid space 176 to treat communicating hydrocephalus or into the ventricle 178 of the brain 174 to treat obstructive hydrocephalus. In some embodiments, the shunt 100 can be positioned partially or entirely under the skin 182. In FIGS. 9-12, as elsewhere herein, the same reference numerals are used to designate features that are the same or similar, except as otherwise noted. Thus, some description of such features is omitted in some instances to avoid repetition.

In some embodiments, for example, as illustrated in FIG. 9, the fluid inlet 104 can be positioned at or near the distal end 150, and the fluid outlet 106 can be positioned between the fluid inlet 104 and the proximal end 152. In some embodiments, the fluid outlet 106 can be positioned between the fluid inlet 104 and stop member 114. The shunt 100 illustrated in FIG. 9 comprises a flow passage 108 connecting the fluid inlet and the fluid outlet. As discussed in greater detail above, the fluid inlet 104, fluid outlet 106, and the flow passage 108 can be configured for flow of cerebrospinal fluid, or other body fluids, therethrough to cancellous bone, trabecular bone, bone marrow, or a medullary cavity. Also as discussed in greater detail above, the movement of bodily fluid through the flow passage 108 can be regulated by one or more valves 120, such as, for example, those illustrated (e.g., in FIGS. 3-6) and/or otherwise disclosed herein.

FIGS. 9 and 10 also show the shunt 100 comprising a tool engagement portion 116 and an interrogation member 154.

As illustrated in FIGS. 10-12, for example, the shunt body 102 can comprise a first shunt body member 156 and a second shunt body member 158. In the shunts 100 illustrated in FIGS. 10-12, the flow passage 108 extends through both shunt body members 156,158. In some embodiments, the flow passage 108 can extend through only one shunt body member. The first shunt body member 156 can comprise the fluid outlet 106 for allowing fluid to pass from the flow passage 108 to a drainage site. The second shunt body member 158 can comprise the fluid inlet 104 for allowing fluid pass from a fluid collection into the flow passage 108.

The second shunt body member 158 can comprise a proximal end 160 and a distal end 162. The fluid inlet 104 can be located at or near an end, e.g., a distal end 162, of the second shunt body member 158. As illustrated in FIG. 9-12, for example, the fluid inlet 104 can comprise a plurality of openings. In some embodiments, the fluid inlet 104 can comprise a single opening. In some embodiments, the fluid inlet 104 can comprise openings positioned to reside in a subarachnoid space 176 and openings positioned to reside in a ventricle 178, for example, as illustrated in FIG. 10. In some embodiments, the fluid inlet 104 can comprise openings positioned to reside in a subarachnoid space 176 without openings positioned to reside in a ventricle 178, or vice versa.

The proximal end 160 of the second shunt body member 158 can be configured for attachment to, engagement with, and/or coupling with the first shunt body member 156. In some embodiments, for example, as illustrated in FIG. 10, the proximal end 160 of the second shunt body member 158 can be configured to be attached to, engaged with, and/or coupled to the proximal end 164 or the distal end 172 of the first shunt body member 156. For example, the proximal end 160 of the second shunt body member 158 and the distal end 172 of the first shunt body member 156 can have complementary shapes in some embodiments. In some embodiments, the first shunt body member 156 and the second shunt body member 158 can be attached to, engaged with, and/or coupled together in series (e.g., FIG. 10) or in parallel (e.g., FIGS. 11 and 12). In some embodiments, for example, as illustrated in FIGS. 11 and 12, the proximal end 160 of the second shunt body member 158 can be configured to be attached to, engaged with, and/or coupled to the proximal end 164 of the first shunt body member 156 at an angle between 0 and 180 degrees, from 45 to 135 degrees, from 80 to 100 degrees, or approximately 90 degrees relative to an axis of the portion of the first shunt body portion 156 extending through bone.

The second shunt body member 158 can have a length between the proximal end 160 and the distal end 162 sufficient to extend from the proximal end 164 of the first shunt body member 156 into a fluid cavity, e.g., a subarachnoid space 176.

In some embodiments, the second shunt body member 158 illustrated in FIG. 8 can comprise multiple components and/or portions comprising different materials. In some embodiments, it can be desirable for all or a portion of the second shunt body member 158 to be formed of a flexible biocompatible material to reduce or avoid injury to the brain 174. For example, in some embodiments, all or a portion of the second shunt body member 158 can be formed of flexible, biocompatible polymer(s) or other materials, or combinations thereof. In some embodiments, all or a portion of the second shunt body member 158 can comprise silicone, plastic, or other material(s), or combinations thereof. In some embodiments, the second shunt body member 158 can have a construction similar to a ventricular catheter. In some embodiments, the second shunt body member 158 can be antibiotic coated.

As discussed in greater detail above, the fluid inlet 104, fluid outlet 106, and the flow passage 108 can be configured for flow of cerebrospinal fluid, or other body fluids, therethrough to cancellous bone, trabecular bone, bone marrow, or a medullary cavity. Also as discussed in greater detail above, the movement of bodily fluid through the flow passage 108 can be regulated by one or more valves 120, such as, for example, those illustrated (e.g., in FIGS. 3-6) and/or otherwise disclosed herein. The valve can form a portion of the flow passage 108 connecting the fluid inlet 104 and the fluid outlet 106, and the valve can regulate the flow of fluid by partially or totally obstructing flow through the valve body. The valve can be positioned partially or wholly within the shunt body 102, in some embodiments, for example, those illustrated in FIGS. 9-11. In some embodiments, the valve can be positioned partially or wholly within one or more components, e.g., the first shunt body member 156 or the second shunt body member 158, of the shunt body. Additionally or alternatively, the valve can be positioned between other components of the shunt body, in some embodiments. In some embodiments, the valve can form a portion of the shunt body, for example, as illustrated in FIG. 12. In some embodiments, the shunt 100 does not comprise any valve.

The foregoing description is provided to enable a person skilled in the art to practice the various configurations described herein. While the subject technology has been particularly described with reference to the various figures and configurations, it should be understood that these are for illustration purposes only and should not be taken as limiting the scope of the subject technology.

There may be many other ways to implement the subject technology. Various functions and elements described herein may be partitioned differently from those shown without departing from the scope of the subject technology. Various modifications to these configurations will be readily apparent to those skilled in the art, and generic principles defined herein may be applied to other configurations. Thus, many changes and modifications may be made to the subject technology, by one having ordinary skill in the art, without departing from the scope of the subject technology.

It is understood that the specific order or hierarchy of steps in the processes disclosed is an illustration of exemplary approaches. Based upon design preferences, it is understood that the specific order or hierarchy of steps in the processes may be rearranged. Some of the steps may be performed simultaneously. The accompanying method claims present elements of the various steps in a sample order, and are not meant to be limited to the specific order or hierarchy presented.

As used herein, the phrase “at least one of” preceding a series of items, with the term “and” or “or” to separate any of the items, modifies the list as a whole, rather than each member of the list (i.e., each item). The phrase “at least one of” does not require selection of at least one of each item listed; rather, the phrase allows a meaning that includes at least one of any one of the items, and/or at least one of any combination of the items, and/or at least one of each of the items. By way of example, the phrases “at least one of A, B, and C” or “at least one of A, B, or C” each refer to only A, only B, or only C; any combination of A, B, and C; and/or at least one of each of A, B, and C.

Terms such as “top,” “bottom,” “front,” “rear” and the like as used in this disclosure should be understood as referring to an arbitrary frame of reference, rather than to the ordinary gravitational frame of reference. Thus, a top surface, a bottom surface, a front surface, and a rear surface may extend upwardly, downwardly, diagonally, or horizontally in a gravitational frame of reference.

Furthermore, to the extent that the term “include,” “have,” or the like is used in the description or the claims, such term is intended to be inclusive in a manner similar to the term “comprise” as “comprise” is interpreted when employed as a transitional word in a claim.

The word “exemplary” is used herein to mean “serving as an example, instance, or illustration.” Any embodiment described herein as “exemplary” is not necessarily to be construed as preferred or advantageous over other embodiments.

A phrase such as “an aspect” does not imply that such aspect is essential to the subject technology or that such aspect applies to all configurations of the subject technology. A disclosure relating to an aspect may apply to all configurations, or one or more configurations. An aspect may provide one or more examples of the disclosure. A phrase such as “an aspect” may refer to one or more aspects and vice versa. A phrase such as “an embodiment” does not imply that such embodiment is essential to the subject technology or that such embodiment applies to all configurations of the subject technology. A disclosure relating to an embodiment may apply to all embodiments, or one or more embodiments. An embodiment may provide one or more examples of the disclosure. A phrase such “an embodiment” may refer to one or more embodiments and vice versa. A phrase such as “a configuration” does not imply that such configuration is essential to the subject technology or that such configuration applies to all configurations of the subject technology. A disclosure relating to a configuration may apply to all configurations, or one or more configurations. A configuration may provide one or more examples of the disclosure. A phrase such as “a configuration” may refer to one or more configurations and vice versa.

A reference to an element in the singular is not intended to mean “one and only one” unless specifically stated, but rather “one or more.” Pronouns in the masculine (e.g., his) include the feminine and neuter gender (e.g., her and its) and vice versa. The term “some” refers to one or more. Headings and subheadings are used for convenience only, do not limit the subject technology, and are not referred to in connection with the interpretation of the description of the subject technology. All structural and functional equivalents to the elements of the various configurations described throughout this disclosure that are known or later come to be known to those of ordinary skill in the art are expressly incorporated herein by reference and intended to be encompassed by the subject technology. Moreover, nothing disclosed herein is intended to be dedicated to the public regardless of whether such disclosure is explicitly recited in the above description.

While certain aspects and embodiments of the subject technology have been described, these have been presented by way of example only, and are not intended to limit the scope of the subject technology. Indeed, the novel methods and systems described herein may be embodied in a variety of other forms without departing from the spirit thereof. The accompanying claims and their equivalents are intended to cover such forms or modifications as would fall within the scope and spirit of the subject technology.

What is claimed is:

1. An intraosseus shunt comprising:
a shunt body configured for insertion into bone and having a fluid inlet, a fluid outlet, and a flow passage extending within the shunt body between the fluid inlet and the fluid outlet, wherein the flow passage, the fluid inlet, and the fluid outlet are configured for cerebrospinal fluid flow therethrough to cancellous bone;
wherein the shunt body comprises a first portion configured to extend through a pedicle of a vertebra, and second portion configured to extend into a subarachnoid space in a spinal canal, the first portion and the second portion coupled together.

2. The shunt of claim 1, wherein when the first and second portions of the shunt are positioned to permit drainage of cerebrospinal fluid from the cavity to the cancellous bone, the first portion and the second portion extend longitudinally along respective axes at a non-zero angle relative to each other.

3. The shunt of claim 1, further comprising a valve attached to the shunt body and configured to control flow through the flow passage.

4. The shunt of claim 1, wherein the shunt has a length of less than 100 mm between a first end and a second end of the shunt.

5. The shunt of claim 1, wherein the shunt body comprises an external surface and a thread on the external surface.

6. The shunt of claim 1, wherein the fluid outlet comprises a plurality of openings positioned between an inlet end and an outlet end of the valve body.

7. The shunt of claim 6, wherein at least a portion of a length of the external surface comprises at least one outlet opening interspersed between thread portions.

8. A method of shunting cerebrospinal fluid from a cerebrospinal fluid cavity to a vertebra, the method comprising:
creating a passage through cortical bone, and into cancellous bone, of the vertebra; and
positioning a shunt such that the cerebrospinal fluid cavity is in communication with the cancellous bone.

9. The method of claim 8, wherein positioning the shunt comprises inserting a first portion of the shunt through a pedicle of the vertebra and through the passage, and inserting a second portion of the shunt into the cerebrospinal fluid cavity.

10. The method of claim 8, wherein the cerebrospinal fluid cavity comprises a subarachnoid space from L1 to L5.

11. The method of claim 8, wherein, when the first and second portions of the shunt are positioned to permit drainage of cerebrospinal fluid from the cavity to the cancellous bone, the first portion and the second portion extend longitudinally along respective axes at a non-zero angle relative to each other.

12. The method of claim 8, wherein positioning the shunt comprises coupling the first portion and the second portion together.

13. The method of claim 8, further comprising inserting a needle between posterior spinous processes of adjacent vertebra and, when the needle abuts a posterior aspect of a vertebra, inserting a dilator and cannula over the needle.

14. The method of claim 8, wherein the cerebrospinal fluid cavity comprises a lumbar cistern.

15. The method of claim 8, wherein the shunt has a passage extending therethrough providing regulated communication between the biological cavity and the cancellous bone controlled by a valve attached to the shunt, the valve configured to permit fluid flow and pressure above a threshold and to inhibit reflux.

16. The method of claim 8, wherein positioning the shunt such that the cerebrospinal fluid cavity is in communication with the cancellous bone comprises:
(i) positioning a first end portion of the shunt in the cancellous bone of the vertebra, the first end portion comprising a fluid outlet; and
(ii) positioning a second end portion of the shunt such that a fluid inlet of the second end portion is in communication with the cerebrospinal fluid cavity.

17. The method of claim 16, wherein positioning a second end portion of the shunt such that a fluid inlet of the second end portion is in communication with the cerebrospinal fluid cavity comprises positioning the second end portion such that the second end portion, including the inlet, resides in a fluid collection.

18. A method of shunting biological fluid from a pressurized biological cavity to a bone, the method comprising:
positioning a first end of a tube in a vertebral body;
positioning a second end of the tube in a subarachnoid space;
wherein the tube has a length of less than 100 mm between the first end and the second end.

19. The method of claim 18, wherein the length of the tube between the first end and the second end is less than 60 mm.

20. The method of claim 18, wherein the length of the tube between the first end and the second end is less than 10 mm.
